(19) United States
(12) Plant Patent Application Publication
(10) Pub. No.: US 2017/0311503 P1
(43) Pub. Date: Oct. 26, 2017

(54) Chinese redbud plant named 'Chongyang'

(50) ;

(71) Applicant: HENAN MINGPIN COLORIZED SEEDLING CO., LTD., Suiping (CN)

(72) Inventor: Huaming Wang, Suiping (CN)

(21) Appl. No.: 15/731,042

(22) Filed: Apr. 11, 2017

(51) Int. Cl. A01H 5/02; A01H 5/12

(52) U.S. Cl. CPC A01H 5/02
USPC PLT/216

(57) ABSTRACT

‘Chongyang’ is a distinctive variety of Chinese redbud plant, which is characterized by its florescence.

Genus and species: Cercis L.

Variety denomination: Chongyang.

### BACKGROUND OF THE INVENTION

The present invention relates to a new and distinct variety of Chinese redbud plant, which is given the varietal denomination of ‘Chongyang’.

In September 2005, a mutated Chinese redbud plant was found among seedlings in Yushan Village, Suiping County, Henan Province, China. The mutated plant bloomed out in both spring as ordinary Chinese redbud plant, and late September. Autumn flowers had a same color as spring flowers, and also had a huge amount. However, autumn florescence was longer than spring florescence. The autumn flowers withered after frost.

In April 2006, branches of the mutated Chinese redbud plant were grafted to a normal Chinese redbud plant through whip grafting. In late September 2006, the grafted plant bloomed again and grew well.

In spring 2007, the grafted plant was asexually reproduced by laying and grafting. Characteristics thereof were all kept.

From 2008-2013, after generations of reproducing, no atavism or further mutation was observed. All generations grew well. Characteristics of the distinct variety were kept stable.

The new and distinct variety of Chinese redbud plant is not commercially available.

### SUMMARY OF THE NEW VARIETY

‘Chongyang’ is a distinct variety of Chinese redbud plant, belonging to deciduous-leaved shrubs and small trees. A whole plant is 2-4 m high. Twigs are coated or glabrous. Leaves are heart-shaped and green, which are glabrous or slightly coated on bottom surfaces. Flowers are densely clustered, butterfly-shaped and rose-red with short pedicels. There are two florescences in a year. A first florescence is from March to April in spring, wherein flowers appear before leaves. A second florescence is from September to November in autumn, wherein the leaves and the flowers exist at the same time. In autumn, a few flowers bloom out in advance, and then all flowers gradually bloom out. Autumn flowers have a same color as spring flowers, and also have a huge amount. However, the autumn florescence is longer than the spring florescence. The autumn flowers wither after frost. Fruits are flat pods. A few fruits are produced in spring while no fruit is produced in autumn.

### BRIEF DESCRIPTION OF THE DRAWINGS

FIG. 1 is a color photograph of the new invention, ‘Chongyang’, showing the plant in the field.

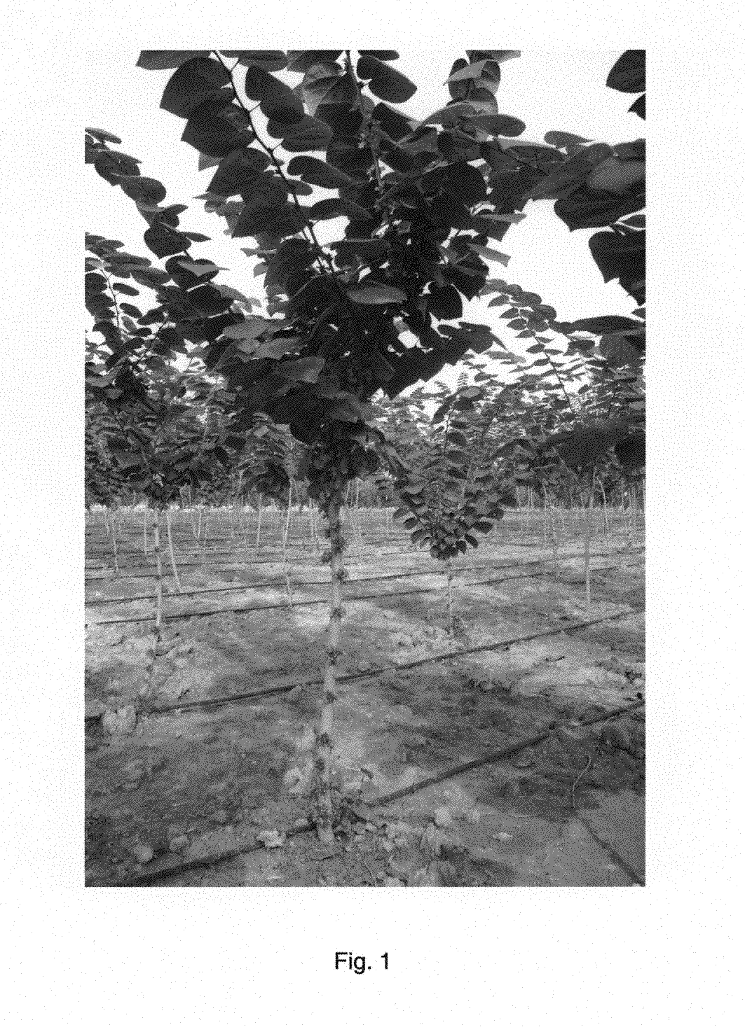

FIG. 2 is a color photograph of the new invention, ‘Chongyang’, showing its stem.

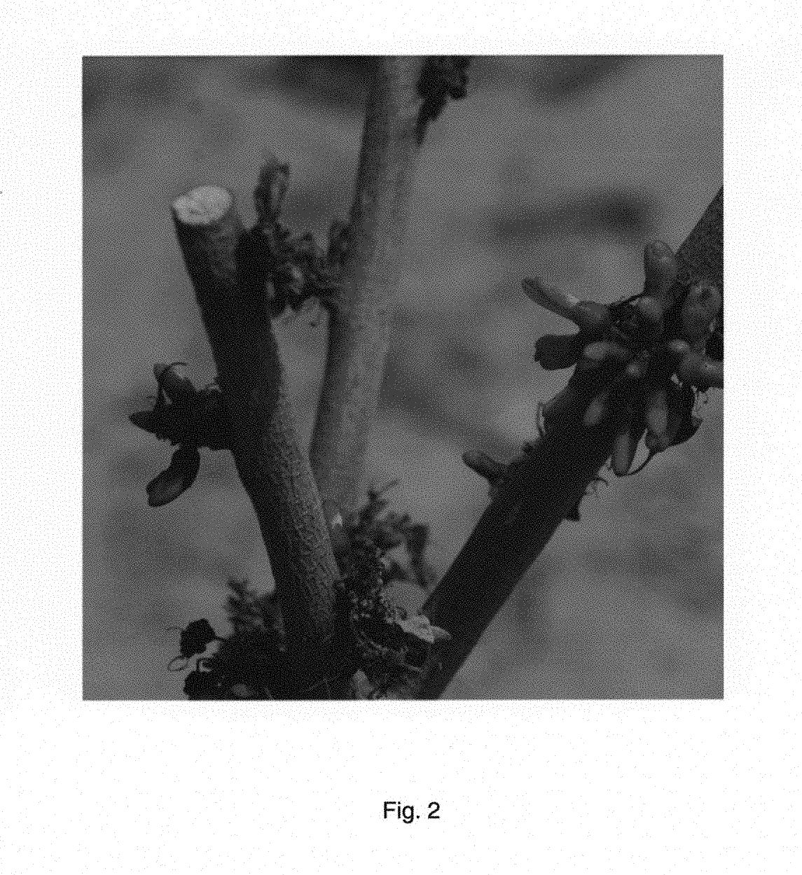

FIG. 3 is a color photograph of the new invention, ‘Chongyang’, showing its leaves.

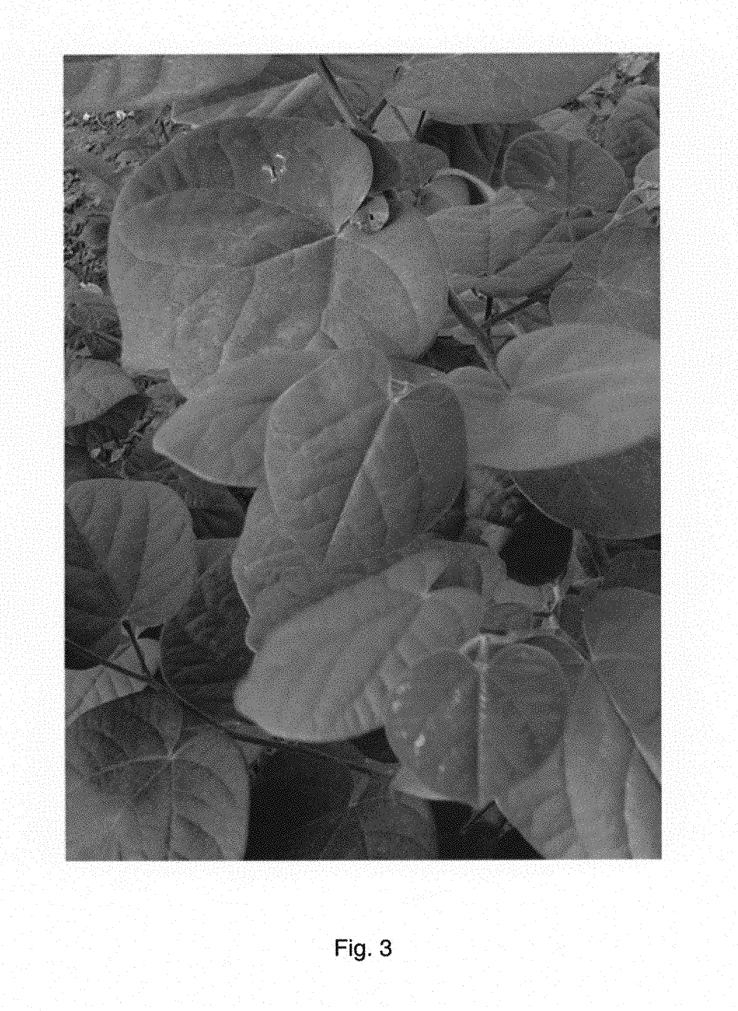

FIG. 4 is a color photograph of the new invention, ‘Chongyang’, showing the appearance of its flowers in spring.

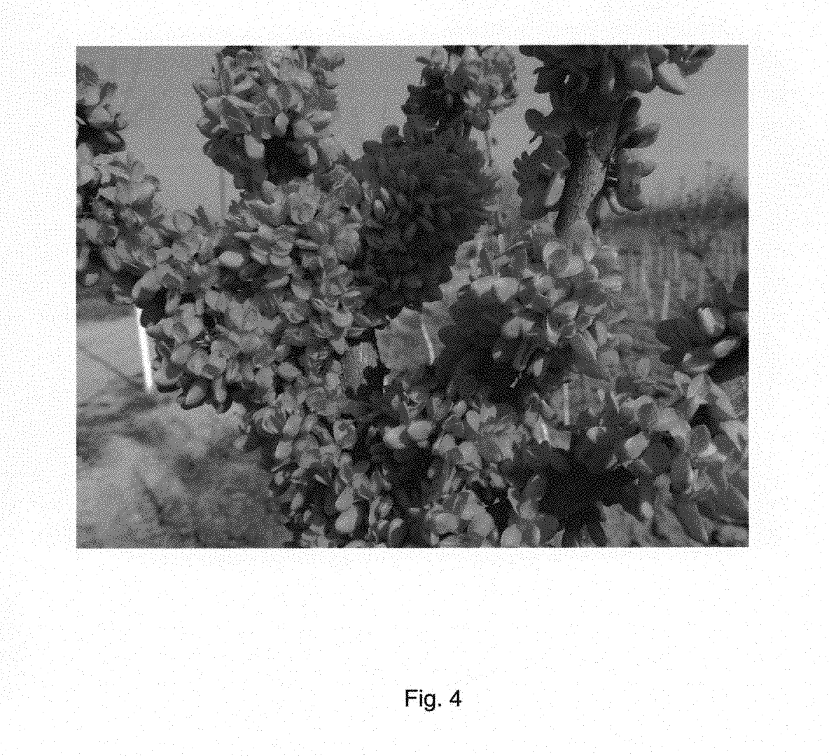

FIG. 5 is a color photograph of the invention, ‘Chongyang’, showing the appearance of its flowers in autumn.

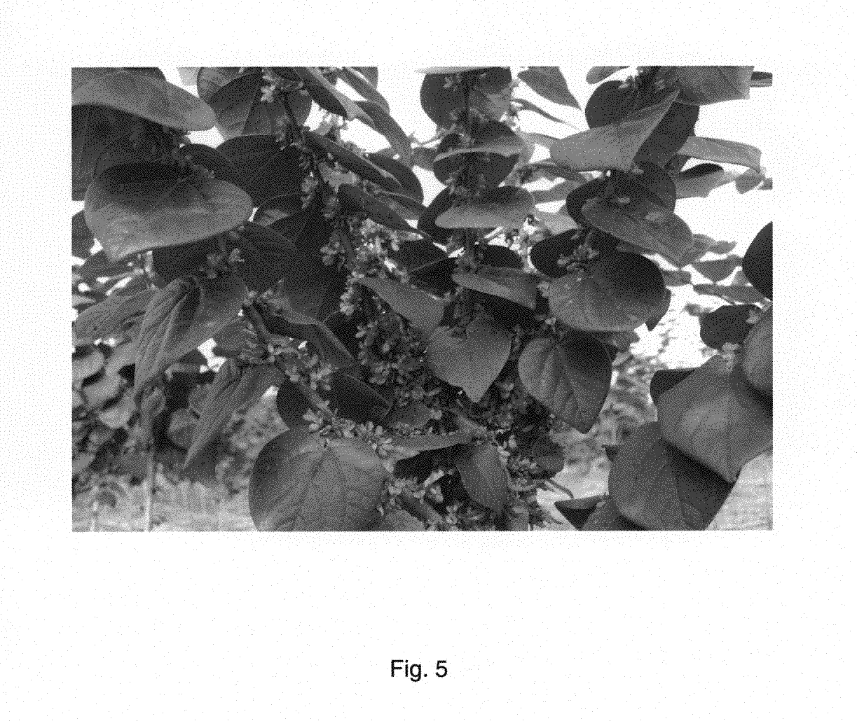

FIG. 6 is a color photograph of the invention, ‘Chongyang’, showing the appearance of its fruits in spring.

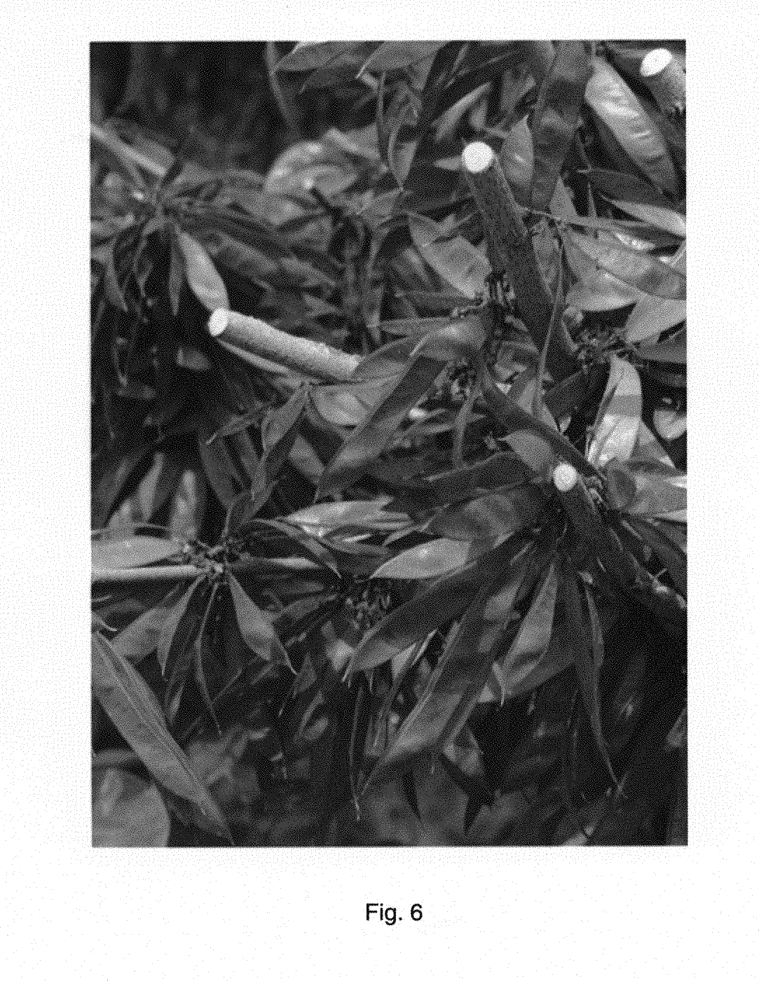

FIG. 7 is a color photograph of the new invention, ‘Chongyang’, showing the plant in the field in spring.

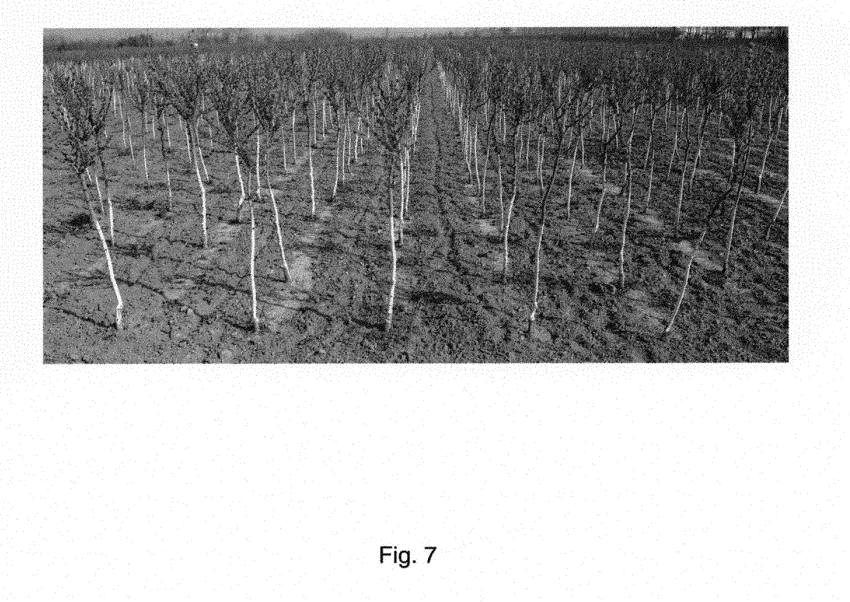

FIG. 8 is a color photograph of the new invention, ‘Chongyang’, showing the plant in the field in autumn.

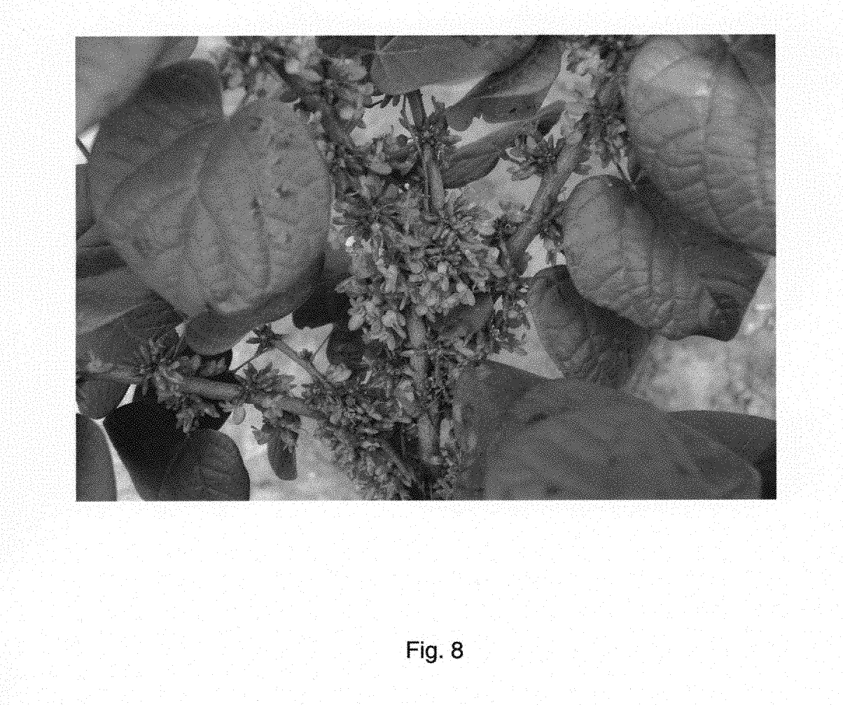

FIG. 9 is a color photograph of the new invention, ‘Chongyang’, showing its seedling in the field.

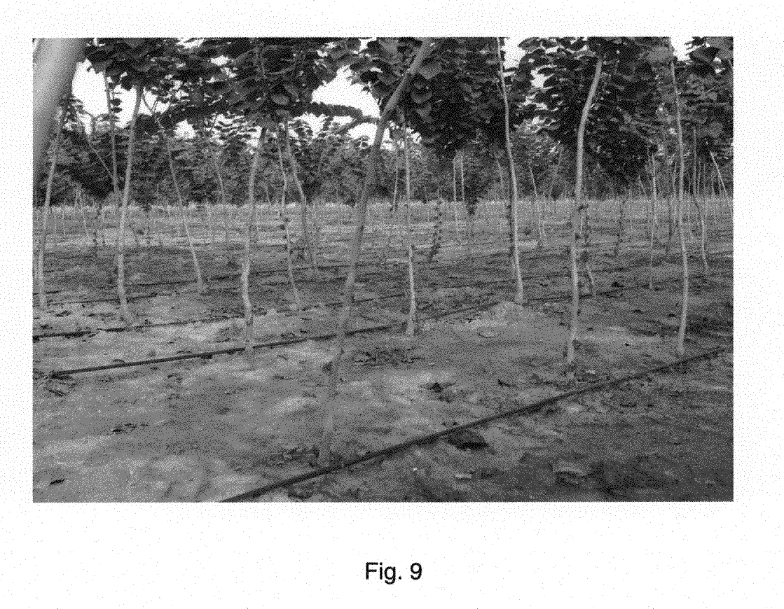

### DETAILED DESCRIPTION

The accompanying colored photographs illustrate the overall appearance of the new and distinct variety of ‘Chongyang’. The descriptions disclosed herein are based upon observations of the plant growth.

‘Chongyang’ is a distinct variety of Chinese redbud plant, belonging to deciduous-leaved shrubs and small trees. A whole plant is 2-4 m high. Twigs are coated or glabrous. Leaves are heart-shaped and green, which are glabrous or slightly coated on bottom surfaces. Flowers are densely clustered, butterfly-shaped and rose-red with short pedicels. There are two florescences in a year. A first florescence is from March to April in spring, wherein flowers appear before leaves. A second florescence is from September to November in autumn, wherein the leaves and the flowers exist at the same time. In autumn, a few flowers bloom out in advance, and then all flowers gradually bloom out. Autumn flowers have a same color as spring flowers, and also have a huge amount. However, the autumn florescence is longer than the spring florescence. The autumn flowers wither after frost. Fruits are flat pods. A few fruits are produced in spring while no fruit is produced in autumn.

‘Chongyang’ is a distinctive variety of Chinese redbud plant, which is able to grow in Hubei, Liaoning, Hebei, Shaanxi, Henan, Gansu, Yunnan, Sichuan and other provinces of China. ‘Choneyang’ is a phototropism species, which has a resistance to minus 23° C. ‘Chongyang’ grows well with fertile and well-drained soil, and is impatient to water logging. The plant also has resistance to sulfur dioxide, hydrogen fluoride and nitrogen, which is suitable for inhaling harmful gases and decorating.

‘Chongyang’ is able to be reproduced by grafting, rameting and laying. Grafting is mainly used, with Chinese redbud plant as a rootstock. Seeds are collected from September to October, and are buried in dry sand covered with shade for wintering. The seeds are sowed in coming late March to early April after germination with warm water.

Whip grafting should be used from April to May while budding should be used from July to September, wherein external branches facing sunshine are preferred for grafting. During dormancy, the branches for grafting should be stored in refrigerator before germination. During growth, the branches should be picked just before grafting. 7 days after budding or 2 weeks after whip, survival rate should be checked and replantation should be prepared if the survival rate is low. Buds of successfully grated plant should be wiped for ensuring nutrients of the grafted branched.

‘Chongyang’ should be cultivated in a high and dry place. There is no strict requirement for soil, but sandy soil which is loose and fertile is preferred. Low-lying areas, which are like to suffer water logging, are not suitable for ‘Chongyang’. Preferably, seedlings transplant should be done in early April.

‘Chongyang’ rarely suffers from diseases. Harmful pests are mainly aphids and longhorned beetles. The former can be eliminated by 1500-2000 times imidacloprid spray, and the latter can be eliminated by green Weilei spray or blocking pest holes with drug.

TABLE
comparison between ‘Chongyang’ and normal Chinese redbud.

| | feature | | |
|---|---|---|---|
| name | Flower feature | | Florescence |
| Chongyang | Spring | flowers appear before | March to April |
| | flowers | leaves | |
| | Autumn | leaves and the flowers | September to |
| | flowers | exist at the same time | November |
| Chinese redbud | Spring | flowers appear before | March to April |
| | flowers | leaves | |

These features and other characteristics of the plant are apparent from the figures.

What is claimed is:

1. A new and distinct variety of Chinese redbud plant named ‘Chongyang’, substantially as described and illustrated.
